(19) United States
(12) Patent Application Publication
(10) Pub. No.: US 2026/0215986 A1
(43) Pub. Date: Jul. 30, 2026

(54) INTEGRATED MULTIFUNCTION TREATMENT CAPSULE

(71) Applicants: Bruno Bafia, Rancho Cucamonga, CA (US); Marcin Lesniak, Losino (PL)

(72) Inventors: Bruno Bafia, Rancho Cucamonga, CA (US); Marcin Lesniak, Losino (PL)

(21) Appl. No.: 19/457,933

(22) Filed: Jan. 23, 2026

Related U.S. Application Data
(60) Provisional application No. 63/749,448, filed on Jan. 24, 2025.

(51) Int. Cl. A61G 10/04; A61M 11/04; A61M 15/00; A61M 21/02; A61N 5/06; A61H 33/06; A61M 21/00

(52) U.S. Cl. CPC A61G 10/04; A61M 11/041; A61M 15/009; A61M 21/02; A61N 5/0613; A61H 2033/061; A61H 33/063; A61H 33/066; A61H 2201/02; A61H 2201/0207; A61H 2201/0221; A61H 2201/0257; A61H 2201/10; A61H 2201/5058; A61M 2021/0016; A61M 2202/064; A61M 2205/051; A61M 2205/502; A61N 2005/0626; A61N 2005/0636; A61N 2005/0659; A61N 2005/0663

(57) ABSTRACT

An integrated multifunction treatment capsule is disclosed for delivering multiple therapeutic modalities within a single enclosed system. The capsule includes an interior enclosure configured to accommodate a human user and a plurality of integrated therapeutic subsystems, including dry salt therapy, wet salt or saline therapy, red-light and near-infrared light therapy, aromatherapy, oxygen therapy, and environmental control. A centralized control unit is operatively coupled to the therapeutic subsystems and configured to selectively activate, coordinate, and regulate operational parameters of the therapies, independently or in combination, during a treatment session. The control unit may further communicate with local or remote user interfaces and networked systems to enable monitoring, customization, and remote control.

### CROSS-REFERENCE TO RELATED APPLICATIONS

This Application claims benefit of the priority filing date of U.S. Provisional Patent Application no. 63/749,448, filed on January 24, 2025, and entitled “Integrated Multifunction Treatment Capsule,” the entire contents of which are hereby incorporated by reference in their entirety.

### FIELD OF THE INVENTION

The present invention relates generally to health, wellness, and therapeutic treatment systems, and more particularly to an integrated, enclosed treatment capsule configured to deliver multiple therapeutic modalities to a human user. The invention specifically pertains to systems and methods for providing coordinated and customizable therapies, including, but not limited to, dry salt therapy, wet salt therapy, red-light and near-infrared light therapy, aromatherapy, oxygen therapy, environmental control, and related wellness treatments within a single multifunctional enclosure under centralized control.

The invention further relates to electronically controlled wellness treatment apparatuses employing programmable interfaces, sensors, and control logic to regulate treatment parameters such as duration, intensity, airflow, particle concentration, illumination wavelength, oxygen delivery, temperature, and humidity, for use in residential, commercial, medical, therapeutic, athletic, and hospitality environments.

### BACKGROUND OF THE INVENTION

Health and wellness treatment technologies such as salt therapy, wet salt or saline therapy, red-light therapy, aromatherapy, and oxygen therapy are widely used to support respiratory health, skin care, relaxation, stress reduction, and overall physical and mental well-being. Traditionally, these therapeutic modalities are delivered using separate devices, dedicated rooms, or independent installations, each designed to perform a single function or limited set of functions.

As a result, existing wellness treatment environments typically require substantial physical space and significant capital investment to accommodate multiple standalone systems. The use of separate devices also increases operational complexity, maintenance requirements, and energy consumption, while limiting accessibility for users and providers. Moreover, the fragmented nature of current solutions often results in a disjointed user experience, requiring users to move between multiple rooms or systems in order to receive different therapies during a single wellness session.

Conventional wellness systems are generally standardized in configuration and operation, offering limited ability to personalize or coordinate treatment parameters across multiple therapeutic modalities. In many cases, therapies are delivered independently rather than in a synchronized manner, and control of environmental conditions, such as airflow, temperature, humidity, and treatment duration, is managed separately by each device. Further, parameters associated with individual therapies, including salt delivery characteristics, oxygen delivery characteristics, light output settings, and aromatic diffusion levels, are typically adjusted in isolation, if adjustable at all. This lack of integration can limit the ability to provide coordinated, repeatable, and customized multi-therapy treatment programs tailored to individual users.

In particular, no commercially available system is capable of delivering dry salt therapy, wet salt therapy, red-light therapy, aromatherapy, and oxygen therapy simultaneously within a single enclosed and unified structure. Existing salt therapy solutions are typically limited to either dry or wet delivery methods and are not integrated with additional therapeutic technologies. Similarly, red-light therapy, aromatherapy, and oxygen therapy are commonly administered using separate equipment that is not designed to operate in a coordinated or enclosed environment.

Accordingly, there exists a need for an integrated wellness treatment system that consolidates multiple therapeutic modalities into a single enclosed capsule, reduces space and operational requirements, and enables centralized control of treatment parameters. Such a system would allow therapies to be delivered independently or in coordinated sequences, simultaneously or according to predefined programs, while providing a personalized, efficient, and repeatable wellness experience. The present invention addresses these and other deficiencies of existing health and wellness treatment technologies.

### SUMMARY OF THE INVENTION

The present invention provides an integrated multifunction treatment capsule configured to deliver a plurality of therapeutic modalities within a single enclosed and unified structure. The capsule defines an interior environment sized to accommodate a human user and houses multiple therapeutic subsystems, including dry salt therapy, wet salt or saline therapy, red-light and near-infrared light therapy, aromatherapy, oxygen therapy, and environmental conditioning features. The therapeutic subsystems are integrated within the enclosure such that each therapy may be delivered independently, sequentially, or simultaneously during a single treatment session, thereby enabling coordinated and customizable wellness experiences that are not achievable using conventional single-modality systems.

In exemplary embodiments, the integrated treatment capsule includes a centralized electronic control system operatively coupled to each therapeutic subsystem and configured to regulate operational parameters associated therewith. Such parameters may include, without limitation, therapy duration, intensity, airflow, salt particle concentration, saline aerosol output, light wavelength and intensity, oxygen concentration and flow rate, temperature, humidity, and sequencing of therapies. The control system enables users or operators to select, customize, store, and repeat predefined or personalized treatment programs with a high degree of consistency and repeatability. By consolidating multiple therapeutic technologies into a single controllable enclosure, the invention reduces space requirements and operational complexity while providing a comprehensive, efficient, and personalized health and wellness treatment solution suitable for residential, commercial, medical, and therapeutic environments.

In an exemplary embodiment, a treatment capsule is provided for delivering therapeutic treatment to a human user. The capsule comprises an enclosed cabin configured to position a human user inside, and a plurality of therapeutic modules coupled to the cabin, including a dry salt generator and a wet salt generator. A control unit is operatively coupled to the therapeutic modules and is configured to selectively activate and control the operation of one or more of the therapeutic modules during a treatment session. The therapeutic modules may further include a red-light therapy module, as well as an aromatherapy module and an oxygen delivery module. The therapeutic modules are operable independently or in coordinated sequences under the control of the control unit. The treatment capsule may further include one or more sensors operatively coupled to the control unit, which provide feedback indicative of at least one operating condition and at least one environmental parameter within the enclosed cabin, such as temperature, humidity, and air quality. A user interface is also provided, operatively coupled to the control unit, and is configured to receive user input relating to the operation of the therapeutic modules. In some embodiments, the user interface is provided via a portable computing device in communication with the control unit.

In an exemplary embodiment, a treatment capsule is provided for salt-based therapeutic treatment. The capsule comprises an enclosed cabin, a dry salt generator configured to deliver dry salt aerosol into the cabin, and a wet salt generator configured to deliver a saline aerosol. A control unit is operatively coupled to the dry and wet salt generators and is configured to operate them selectively, sequentially, or simultaneously during a treatment session. The dry and wet salt generators may be fluidly coupled to the cabin through respective conduits terminating in outlet nozzles disposed within the enclosed cabin. The capsule may further include an air ventilator configured to control airflow within the cabin during delivery of salt aerosols. The control unit is configured to regulate salt concentration, aerosol flow rate, and treatment duration. Additionally, the capsule may include an aromatherapy module configured to introduce aromatic substances into the cabin, with the aromatherapy module comprising a plurality of aromatic oil diffusers independently controllable by the control unit. A red-light therapy module may also be included, configured to emit red and near-infrared light, with the control unit controlling the wavelength, intensity, and duration of light emitted.

In an exemplary embodiment, a treatment capsule is provided for coordinated therapeutic treatment of a human user. The capsule comprises an enclosed cabin and a plurality of therapeutic subsystems, including a dry salt therapy subsystem, a wet salt therapy subsystem, a light therapy subsystem, an aromatherapy subsystem, and an oxygen therapy subsystem. A control unit is operatively coupled to each therapeutic subsystem and is configured to coordinate operation of two or more subsystems during a single treatment session. The control unit may deliver at least two therapeutic subsystems simultaneously within the cabin and may execute a predefined or user-customizable treatment program comprising sequential activation of the therapeutic subsystems. Furthermore, the control unit may communicate with a remote station via a network to enable remote monitoring, configuration, and control of the treatment capsule.

### BRIEF DESCRIPTION OF THE FIGURES

FIG. 1 is an exterior perspective view of an exemplary multifunctional treatment capsule according to an embodiment of the present invention.

FIG. 2 is a rear-left perspective view of an interior cabin of the multifunctional treatment capsule with an exterior enclosure removed, illustrating representative therapy components.

FIG. 3 is a front-left perspective view of the multifunctional treatment capsule with the exterior enclosure removed, illustrating representative therapy and environmental control components.

FIG. 4 is an exploded rear-left perspective view of the multifunctional treatment capsule with the exterior enclosure removed, illustrating removable therapy components.

FIG. 5 is a partial interior perspective view of a front portion of the interior cabin of the multifunctional treatment capsule, illustrating interior therapy and airflow components.

FIG. 6 is a partial interior perspective view of a rear portion of the interior cabin of the multifunctional treatment capsule, illustrating interior therapy delivery components.

FIG. 7 is a block diagram illustrating functional relationships among control, therapy, sensing, and communication components of the multifunctional treatment capsule.

FIG. 8 is a block diagram of an exemplary control unit according to an embodiment of the present invention.

### DETAILED DESCRIPTION OF THE INVENTION

The present invention is described more fully hereinafter, but not all embodiments are shown. While the invention has been described with reference to exemplary embodiments, it will be understood by those skilled in the art that various changes may be made, and equivalents may be substituted for elements thereof without departing from the scope of the disclosure. ln addition, many modifications may be made to adapt a particular structure or material to the teachings of the disclosure without departing from the essential scope thereof.

The drawings accompanying the application are for illustrative purposes only. They are not intended to limit the embodiments of the present application. Additionally, the drawings are not drawn to scale. Common elements between different figures may retain the same numerical designation.

The present invention relates to an integrated multifunction treatment capsule configured to deliver multiple therapeutic modalities within a single enclosed system. The integrated multifunction treatment capsule is designed to overcome the limitations of conventional single-modality wellness devices by consolidating a plurality of therapeutic subsystems into a unified enclosure capable of providing coordinated, personalized, and repeatable treatment sessions.

In exemplary embodiments, the integrated multifunction treatment capsule defines an enclosed interior sized to accommodate a human user in a seated, reclined, or partially reclined position. The enclosure houses a plurality of therapeutic subsystems, which may include, but are not limited to, dry salt aerosol delivery, wet salt or saline aerosol delivery, red-light and near-infrared light therapy, aromatherapy diffusion, oxygen enrichment or delivery, air purification and filtration, humidity control, temperature control, sound therapy, and guided breathing or relaxation protocols. The therapeutic subsystems are integrated within the enclosure in a manner that allows efficient delivery of one or more therapies to the user during a single treatment session, during successive treatment sessions, or as part of a predefined, repeatable, or customizable treatment protocol executed over time.

Each therapeutic subsystem of the integrated multifunction treatment capsule may be configured to operate independently or in coordinated sequences with one or more other therapeutic subsystems. In some embodiments, multiple therapeutic modalities may be delivered simultaneously, while in other embodiments the therapeutic subsystems may be activated sequentially according to a predetermined treatment program. This flexible operational architecture enables selective combination and coordination of therapies based on individual user preferences or therapeutic objectives.

The integrated multifunction treatment capsule further includes a control system operatively coupled to the therapeutic subsystems and configured to regulate operational parameters associated with each therapy. Such parameters may include, but are not limited to, therapy duration, therapy intensity, airflow characteristics, salt particle concentration, saline aerosol output, light wavelength and intensity, oxygen flow or concentration, and environmental conditions within the enclosure, including temperature and humidity. The control system may coordinate these parameters across multiple therapeutic subsystems to provide controlled and repeatable treatment protocols.

In exemplary embodiments, the control system may include manual controls, digital interfaces, touchscreen displays, software-based programming, remote operation capability, or network-connected functionality. These features allow users or operators to select, customize, store, and repeat personalized treatment programs with a high degree of consistency. The control system may further provide monitoring and status feedback to support reliable operation of the integrated multifunction treatment capsule.

The integrated multifunction treatment capsule may be constructed using modular components to facilitate scalability, customization, maintenance, and future upgrades. Modular construction allows therapeutic subsystems to be added, removed, or replaced without substantial modification to the enclosure. The system is suitable for deployment in a variety of environments, including, but not limited to, wellness centers, spas, medical and therapeutic facilities, gyms, hotels, resorts, athletic training facilities, professional sports organizations, transportation hubs, and residential settings.

By integrating multiple therapeutic modalities within a single enclosed and controllable system, the integrated multifunction treatment capsule reduces space and operational requirements, lowers overall system costs, enhances treatment personalization, and provides a coordinated wellness experience relative to conventional single-modality devices.

In an exemplary embodiment, the present invention provides a multifunctional health and treatment capsule that integrates wet salt therapy, dry salt therapy, red-light and near-infrared light therapy, aromatherapy, and oxygen therapy within a single enclosed and unified structure. Each therapeutic modality may be delivered individually or in any combination during a single treatment session under centralized control. The integration of multiple therapeutic technologies into a single multifunctional enclosure enables users to selectively combine, customize, and coordinate therapies in a streamlined and efficient manner. The capsule may be configured for residential or commercial use and provides advanced therapeutic capabilities in a convenient, user-friendly environment designed to reduce overall treatment time while maximizing therapeutic efficiency.

In exemplary embodiments, the treatment capsule is configured to simultaneously or sequentially target respiratory function, skin health, mental clarity, and emotional balance during a single treatment session. Dry salt therapy, also referred to as halotherapy, is provided by dispersing micronized dry salt particles into the air within the enclosed interior to create a controlled microclimate resembling that of natural salt caves or salt mines. Wet salt therapy, also referred to as saline or brine therapy, is delivered by aerosolizing a saline solution using a misting or nebulizing device, wherein water acts as a carrier medium for salt particles. The inclusion of both dry salt therapy and wet salt therapy subsystems within the same capsule enables delivery of distinct salt-based therapeutic modalities within a shared enclosed environment, either independently or in coordinated operation.

In some implementations, dry salt therapy is provided by a dry salt generator coupled to the interior of the treatment capsule and configured to mechanically grind, mill, or abrade pharmaceutical-grade sodium chloride into micron-sized particles. The dry salt generator may further include a dispersion mechanism, such as a fan or air propulsion system, that entrains the micronized salt particles into a controlled airflow and distributes them uniformly throughout the enclosed cabin. The generator is configured to regulate particle size, concentration, and flow rate to maintain a consistent dry salt microclimate within the interior during operation, wherein related operating parameters are controllable by a processing control unit.

In some implementations, wet salt therapy is provided by a wet salt generator coupled to the interior of the treatment capsule and configured to aerosolize a saline or brine solution. The wet salt generator may include a reservoir for holding the saline solution and a misting, ultrasonic, or nebulizing device that converts the solution into fine droplets suspended in air. The system may be configured to control droplet size, humidity, and saline concentration, thereby enabling delivery of a breathable saline mist into the enclosed cabin for therapeutic exposure, either alone or in combination with dry salt therapy, wherein related operating parameters are controllable by a processing control unit.

The integrated treatment capsule can further include a red-light therapy subsystem configured to emit non-invasive wavelengths of red and near-infrared light, which in exemplary embodiments may include wavelengths of approximately 660 nanometers and approximately 850 nanometers. Such light therapy is configured to promote cellular health, tissue regeneration, joint recovery, and muscle recovery, and may assist in reducing pain, inflammation, and stiffness associated with muscular, joint, or tissue conditions. The red-light therapy subsystem may be configured to control treatment parameters including wavelength selection, light intensity, exposure duration, and duty cycle to deliver a targeted photobiomodulation effect. In some implementations, the red-light therapy subsystem includes one or more light sources mounted within the interior of the capsule to provide uniform or localized illumination. Related operating parameters of the red-light therapy subsystem are controllable by a processing control unit.

The integrated treatment capsule can further include an oxygen therapy subsystem configured to enrich or regulate the breathing environment within the enclosure to promote relaxation, revitalization, and improved breathing efficiency. In some implementations, oxygen therapy is provided by an oxygen generator, oxygen concentrator, or compressed oxygen tank operably connected to the interior of the capsule and configured to deliver oxygen-enriched air into the enclosed breathing space. The oxygen therapy subsystem may be configured to control parameters including oxygen concentration, flow rate, delivery duration, and timing relative to other therapeutic modalities. Oxygen may be delivered continuously or intermittently through one or more outlets positioned within the capsule to promote even distribution. Related operating parameters of the oxygen therapy subsystem are controllable by a processing control unit.

Additionally, an aromatherapy subsystem may be configured to deliver aromatic substances into the enclosed interior of the treatment capsule to promote physical, mental, and emotional well-being. In exemplary embodiments, the aromatherapy subsystem utilizes natural plant extracts, including essential oils, which may be administered through inhalation using airflow-based diffusion, vapor-based diffusion, steam-based delivery, or combinations thereof. The aromatherapy subsystem may be operatively coupled to the control system to regulate delivery parameters such as oil selection, diffusion rate, concentration, timing, and duration.

Aromatherapy provided by the treatment capsule may deliver therapeutic effects including, but not limited to, relaxation, stress and anxiety reduction, mood enhancement, improved sleep quality, pain management, improved digestion, immune system support, and overall emotional balance. In exemplary embodiments, aromatherapy delivery may be tailored to individual user preferences or therapeutic objectives through selection of specific aromatic compounds and adjustable delivery settings. The aromatherapy subsystem may operate independently or in coordination with one or more additional therapeutic subsystems of the treatment capsule during a treatment session.

In some implementations, the aromatherapy subsystem is provided by one or more aromatic oil diffusers coupled to the interior of the treatment capsule and configured to disperse aromatic substances into the enclosed environment. By way of example, the diffuser may include an ultrasonic diffuser that uses high-frequency vibrations to atomize a liquid containing one or more essential oils into a fine mist without the application of heat. In other implementations, the diffuser may include a nebulizing diffuser, a fan-assisted diffuser, or a vaporizing diffuser configured to deliver aromatic compounds in a controlled manner. The one or more diffusers may be positioned to promote uniform distribution of aromatic substances within the capsule and may be configured to operate continuously or intermittently during a treatment session. Delivery parameters including oil selection, diffusion rate, concentration, timing, and duration may be adjustable, wherein related operating parameters of the aromatherapy subsystem are controllable by a processing control unit.

In some implementations, the integrated treatment capsule includes a controllable reclining seat coupled to the interior cabin and configured to support a user in one or more seated or reclined positions during a treatment session. The reclining seat may include one or more heating features, such as a heated mat, heating pad, resistive heating elements, or embedded heating coils integrated into a seat base, backrest, or leg support, and configured to provide localized or full-body warmth. The reclining seat may further include one or more massage features provided by massage components coupled to the seat, which may include mechanical actuators, vibrating elements, rollers, or pneumatic massage elements such as air bladders configured to inflate and deflate to apply pressure to selected regions of a user’s body. In exemplary embodiments, the reclining position, heat output, massage intensity, massage pattern, and duration may be adjustable to accommodate user comfort and therapeutic preferences.

In combination, the integrated delivery of salt-based therapies, light therapy, oxygen therapy, and aromatherapy within a single enclosed capsule provides synergistic therapeutic benefits that may include respiratory relief, skin care, detoxification, relaxation, stress reduction, improved circulation, immune system support, enhanced mental clarity, and emotional balance. The ability to coordinate and customize these therapies within a single system allows the treatment capsule to provide a comprehensive, repeatable, and personalized treatment experience that is not achievable using conventional standalone or single-modality wellness devices.

In exemplary embodiments, the integrated multifunction treatment capsule employs a centralized control architecture configured to enable rapid setup, efficient operation, and simplified maintenance of the therapeutic subsystems. Centralized control reduces system complexity and downtime by coordinating operation of multiple therapeutic modalities through a unified hardware and software framework. Such architecture facilitates streamlined system diagnostics, software updates, and subsystem management, thereby enhancing overall reliability and service availability.

The control system may include a user-facing interface accessible via a tablet, touchscreen, or similar digital device, allowing users to easily select, activate, deactivate, or combine one or more therapeutic modalities during a treatment session. The interface may support customizable treatment settings that enable therapies to be tailored to individual health and wellness preferences or objectives. In exemplary embodiments, the interface presents a clean and intuitive layout with visual indicators, guided prompts, and informational tooltips to assist users in navigating available treatment options, therapy combinations, and customization levels.

In some embodiments, technical aspects of system configuration and operation may be managed by an operator, technician, or staff member. A comprehensive control panel may display user selections, subsystem status, and corresponding operational parameters in real time. The control system may guide the operator through step-by-step setup procedures, including automated verification routines configured to confirm proper deployment, calibration, and functionality of selected therapeutic subsystems prior to or during operation.

The control system may further be configured to monitor system performance in real time and generate alerts, notifications, or status messages in response to detected conditions, such as parameter deviations, maintenance requirements, or subsystem faults. Such monitoring capabilities allow operators to proactively maintain service quality, ensure user safety and comfort, and promptly address user inquiries or operational issues. The centralized control architecture thereby supports efficient management, scalability, and consistent delivery of coordinated wellness treatments within the integrated multifunction treatment capsule.

The multifunctional treatment capsule may be constructed from a variety of structural and functional materials selected to provide durability, user comfort, aesthetic appeal, and compatibility with the integrated therapeutic subsystems. Such materials may include, but are not limited to, fiberglass, metals, composite materials, polymers, glass, leather or synthetic upholstery materials, acoustic materials, and electronic and electrical components. The materials may be selected to withstand exposure to moisture, salt particles, light emissions, airflow, temperature variations, and repeated use in residential or commercial environments.

In exemplary embodiments, the exterior surface of the capsule is molded from fiberglass to provide a lightweight, rigid, and corrosion-resistant enclosure. Fiberglass construction allows for smooth, contoured exterior and interior surfaces that enhance structural integrity and ease of cleaning. The molded fiberglass exterior may further support integration of access panels, lighting elements, ventilation openings, control interfaces, and mounting features for internal therapeutic subsystems. In some embodiments, the exterior may be finished with protective coatings, decorative surfaces, or antimicrobial treatments to enhance durability and hygiene.

Internal structural components, mounting frames, or subsystem housings may be formed from metal or reinforced composite materials to provide mechanical strength and thermal stability. Interior user-contact surfaces may incorporate padded elements, seating structures, or liners formed from leather, synthetic leather, or other comfort-enhancing materials. Electronic components, sensors, wiring, and control modules may be housed within dedicated compartments to protect sensitive equipment and facilitate maintenance, upgrades, or replacement.

In exemplary embodiments, the materials and components used in construction of the multifunctional treatment capsule are selected to satisfy applicable safety, fire-resistance, and durability requirements for residential and commercial installations. Structural, interior, and electronic components may be formed from materials that are flame-retardant, fire-resistant, or self-extinguishing, and may comply with applicable electrical, building, and safety standards. Electrical wiring, control circuitry, lighting elements, and heating or airflow components may be insulated, shielded, and isolated to reduce fire risk and enhance operational safety during use.

Additionally, material selection and surface treatments may be configured to mitigate corrosion, degradation, or performance loss resulting from prolonged exposure to salt particles, saline aerosols, humidity, and other environmental conditions associated with salt-based therapies. In exemplary embodiments, interior and structural components may be fabricated from corrosion-resistant metals, coated composites, or polymer-based materials, and may include protective finishes, sealants, or barriers to inhibit salt accumulation and moisture penetration. Such corrosion-mitigating features enhance long-term durability, reduce maintenance requirements, and preserve reliable operation of therapeutic subsystems, airflow pathways, and electronic components within the enclosed environment.

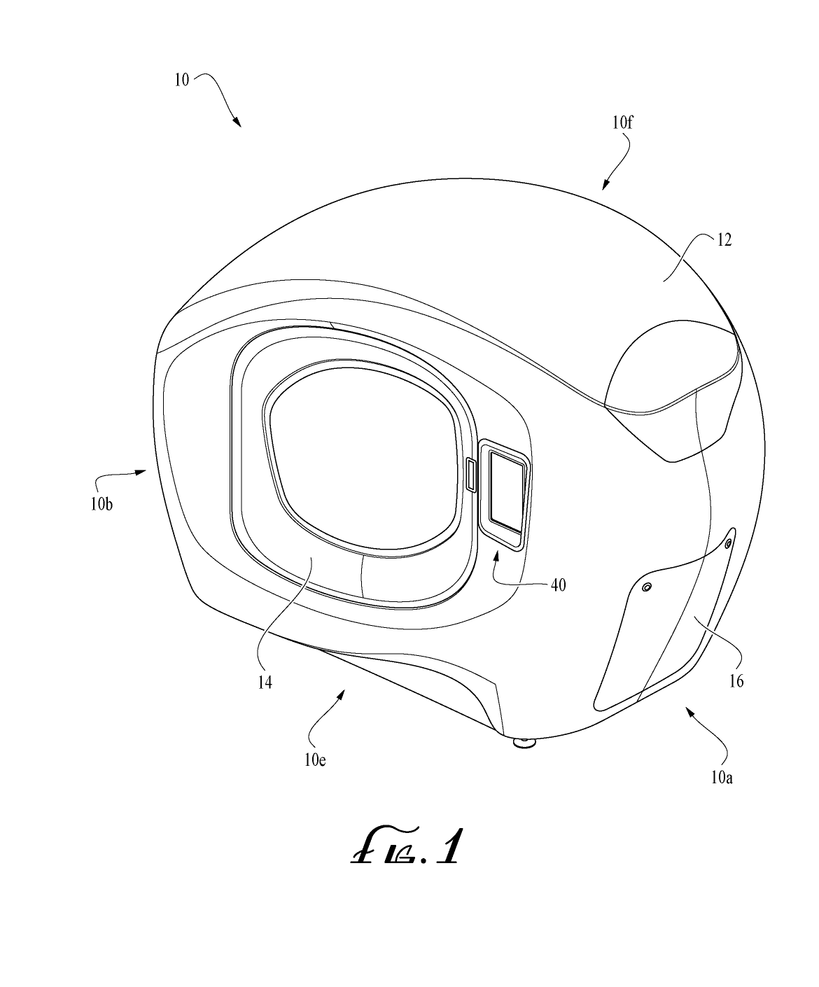

FIGS. 1–6 illustrate an embodiment of the invention. Various features of this embodiment are explained based on the foregoing general description of the invention and are not repeated here to avoid redundancy.

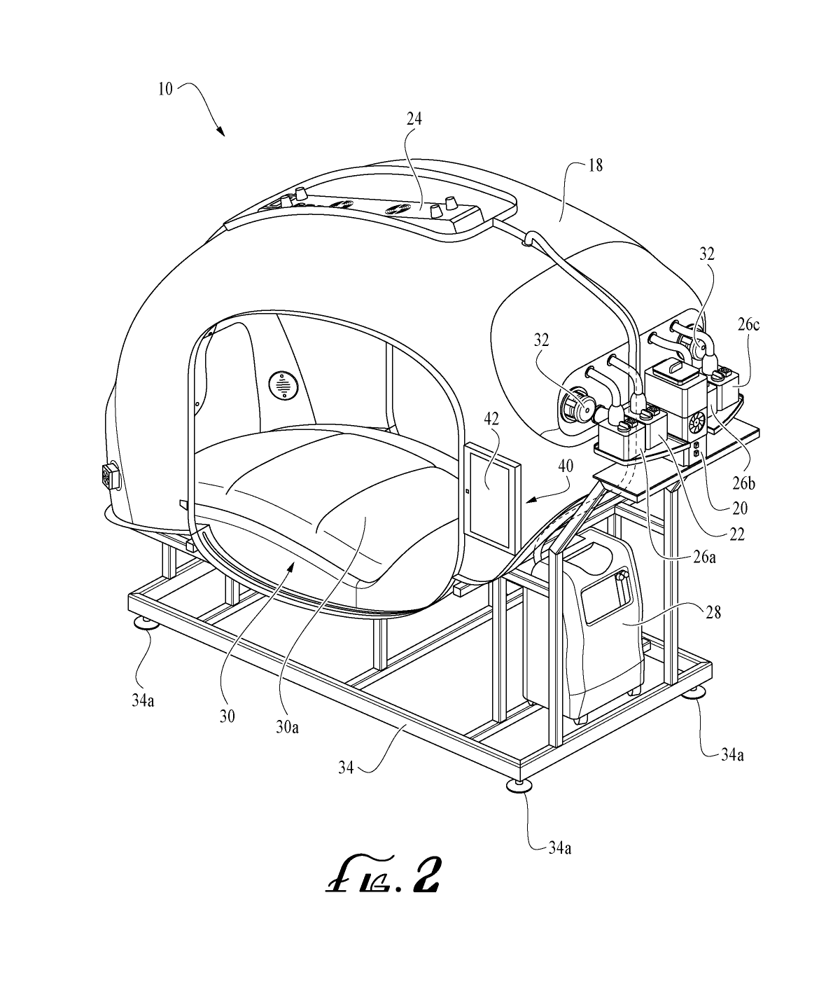

Referring to FIG. 1, the figure illustrates an exterior view of a multifunctional treatment capsule 10 (also referred to herein as capsule 10), showing a fully enclosed exterior cabin 12, an entrance panel 14, a control unit 40, and one or more service or equipment compartments 16. The exterior cabin 12 houses an interior frame or interior cabin (18 as shown in FIG. 2) and various subsystems, components, or therapy modules that are not shown in this figure.

The capsule 10 includes a rear side 10a, a front side 10b, a top side 10c, a bottom side 10d, a left side 10e, a right side 10f, and a bottom side, wherein the entrance panel 14 is coupled to the left side 10e of the capsule 10 as shown. In other implementations, the entrance panel 14 may be coupled to any other side of the capsule 10, may be provided as a hinged door, sliding door, removable panel, or lift-up panel, or more than one entrance panel may be provided.

The entrance panel 14 is configured to allow a user to enter and exit the capsule 10 and may include sealing elements, latches, hinges, handles, transparent or translucent portions, and safety interlocks. In exemplary embodiments, the entrance panel 14 cooperates with the exterior cabin 12 and the interior cabin (18 as shown in FIG. 2) to form a substantially enclosed interior environment when closed.

In operation, a user enters the capsule 10 through the entrance panel 14 and positions themselves within the capsule 10 to receive one or more therapeutic treatments. The desired treatment may be completely selected, adjusted, and modified by the control unit 40, and may be limited to a single therapy module disposed within the capsule 10 or may comprise a combination of multiple therapy functions performed simultaneously, sequentially, or in a predetermined order. Parameters associated with each treatment or therapy, such as the duration, intensity, airflow, concentration, temperature, and sequencing, may be determined by the user, by an operator, or automatically by predefined or adaptive treatment programs executed by the control unit 40.

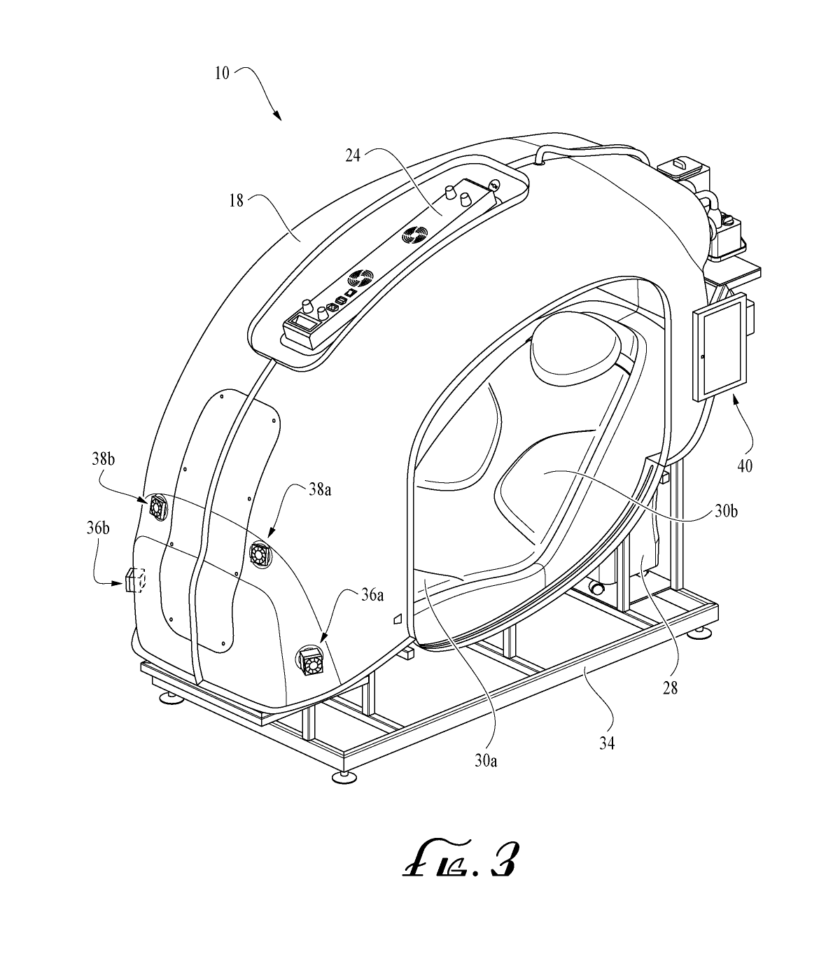

Referring to FIG. 2, the figure illustrates a rear-left perspective view of the interior frame or interior cabin 18, with the exterior cabin 12 removed. As shown, various therapy modules and components are coupled to the interior cabin 18, including a dry salt generator 20 configured to provide dry salt therapy, a wet salt generator 22 configured to provide wet salt or saline therapy, a red-light therapy module 24 configured to provide one or more red-light sources, an aromatherapy module comprising one or more aromatic oil diffusers 26a, 26b, 26c (collectively 26), an oxygen generator or oxygen tank 28 configured to provide oxygen therapy, a reclining seat 30 having integrated heating and massage features 30a (and 30b as shown in FIG. 3), one or more speakers 32 configured to provide sound or music therapy, and a stand 34.

All therapy modules or components are electronically coupled to the control unit 40, wherein such coupling may be provided through wired connections, wireless connections, or combinations thereof. The oxygen generator or tank 28 may be coupled to the stand 34, to the interior cabin 18, or to both.

The dry salt generator 20, the wet salt generator 22, the aromatherapy module (26a, 26b, 26c), and the oxygen generator or tank 28 are operably connected to the interior of the interior cabin 18 through one or more connecting pipes or hoses, as described in further detail below.

The control unit 40 further comprises or is operatively coupled to a user-interface display 42 for interaction with a user or patient.

The interior cabin 18 is supported by the stand 34, which is configured to support the capsule 10 on a surface. The stand 34 may include a plurality of legs 34a, which may be fixed or adjustable. In exemplary embodiments, the legs 34a allow leveling, height adjustment, vibration isolation, or alignment of the capsule 10 on uneven or varying surfaces such as floors or platforms.

Referring to FIG. 3, the figure illustrates a front-left perspective view of the capsule 10 with the exterior cabin 12 removed. As shown, the interior cabin 18 is further coupled to a heating module (36a, 36b) configured to heat the interior of the interior cabin 18 to a predetermined temperature, and a ventilation module (38a, 38b) configured to control airflow within the interior cabin 18. All modules are connected to the control unit 40 through wired or wireless connections.

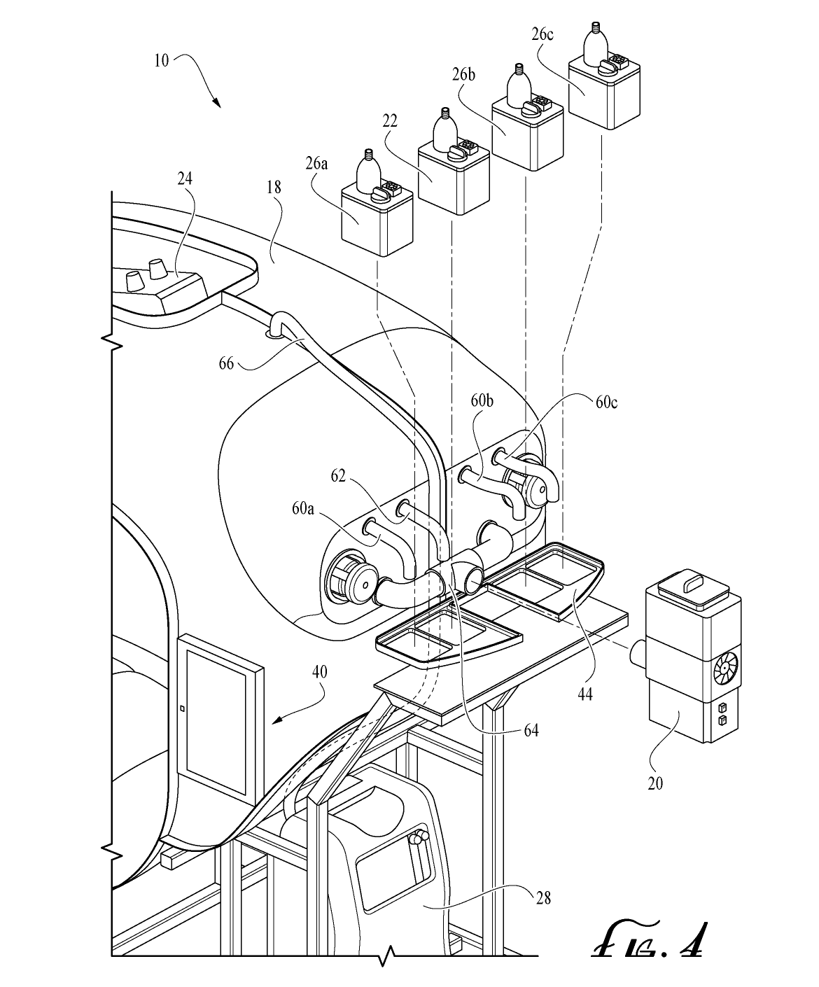

Referring to FIG. 4, the figure illustrates an exploded rear-left perspective view of the capsule 10 with the exterior cabin 12 removed, and may be viewed in conjunction with FIG. 2. As shown, the dry salt generator 20, the wet salt generator 22, and the aromatherapy module (26a, 26b, 26c) is coupled to the interior cabin 18 through a removable tray 44, wherein the modules may be easily attached to or detached from the tray 44 for maintenance, replacement, or upgrading.

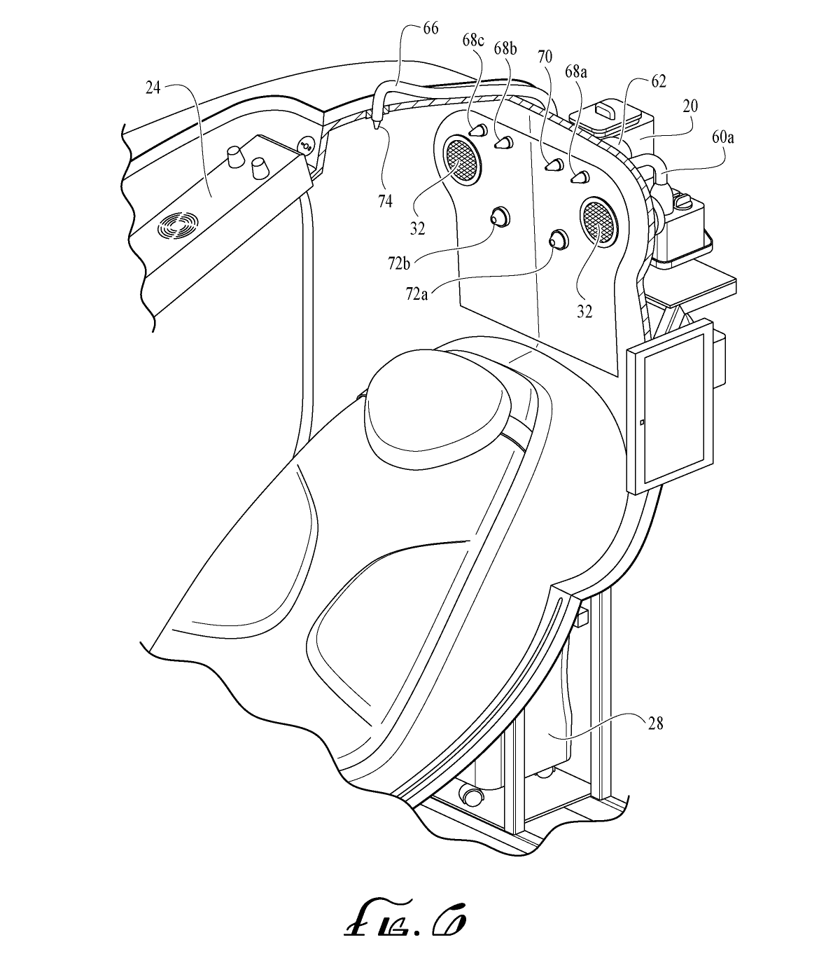

The aromatherapy module 26 comprises one or more aromatic oil diffusers 26a, 26b, 26c, each of which is connected to the interior cabin 18 through respective connecting pipes or hoses 60a, 60b, 60c. Each connecting pipe or hose 60a–60c is connected to a corresponding outlet nozzle disposed inside the interior cabin 18, as shown in FIG. 6.

The wet salt generator 22, which may include a saline tank and misting or nebulizing device, is coupled to the interior cabin 18 through a connecting pipe or hose 62, which is connected to a corresponding nozzle within the interior cabin 18, as shown in FIG. 6.

The dry salt generator 20 is coupled to the interior cabin 18 through a bifurcated connecting pipe or hose 64, which is connected to corresponding nozzles within the interior cabin 18.

Similarly, the oxygen generator or tank 28 is coupled to the interior cabin 18 through a connecting pipe or hose 66, which is connected to a corresponding nozzle inside the interior cabin 18.

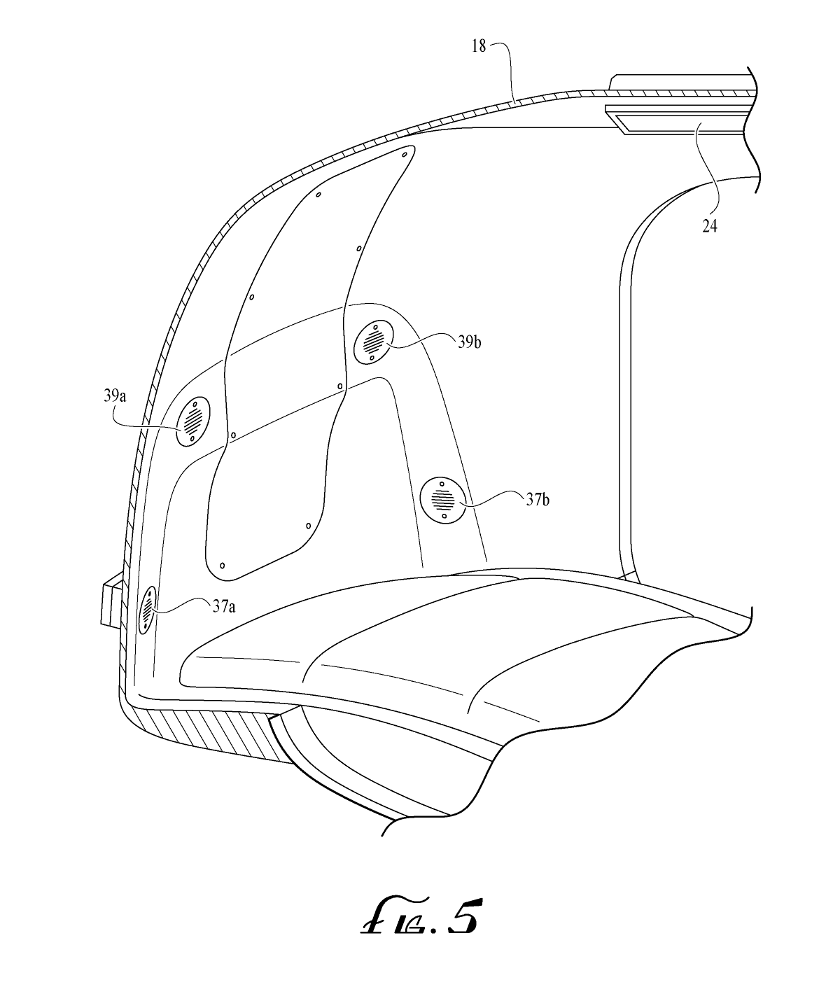

Referring to FIG. 5, the figure illustrates a sliced perspective interior view of the front side of the interior cabin 18 of the capsule 10. Viewed in conjunction with FIG. 3, FIGS. 3 and 5 show air outlets (37a, 37b) of the heating module (36a, 36b), and air inlets or outlets (39a, 39b) of the ventilation module (38a, 38b). These modules are electronically connected to the control unit 40 to provide desired airflow characteristics and temperature conditions within the interior cabin 18.

The figure further shows the red-light therapy module 24 from inside the capsule 10, wherein the red-light sources of the the red-light therapy module are configured to provide red-light and/or near-infrared light therapy.

Referring to FIG. 6, the figure illustrates a sliced perspective interior view of the rear side of the interior cabin 18 of the capsule 10.

Viewed in conjunction with FIG. 4, FIGS. 4 and 6 illustrate how the connecting pipes or hoses are coupled to the interior cabin 18 through corresponding outlet nozzles.

In particular, the aromatic oil diffusers 26a, 26b, 26c are connected to the interior cabin 18 through the connecting pipes or hoses 60a, 60b, 60c, respectively. Each connecting pipe or hose 60a–60c is connected at one end to a respective aromatic oil diffuser 26a–26c as shown in FIG. 4, and at the other end to a corresponding outlet nozzle 68a, 68b, 68c disposed within the interior cabin 18, as shown in FIG. 6.

The wet salt generator 22 is connected to the interior cabin 18 through the connecting pipe or hose 62, which is connected at one end to the wet salt generator 22 and at the other end to a corresponding outlet nozzle 70.

The dry salt generator 20 is connected to the interior cabin 18 through the bifurcated connecting pipe or hose 64 (as shown in FIG. 4), which is connected at one end to the dry salt generator 20 and at two opposing ends to corresponding outlet nozzles 72a and 72b.

Similarly, the oxygen generator or tank 28 is connected to the interior cabin 18 through the connecting pipe or hose 66, which terminates at a corresponding outlet nozzle 74.

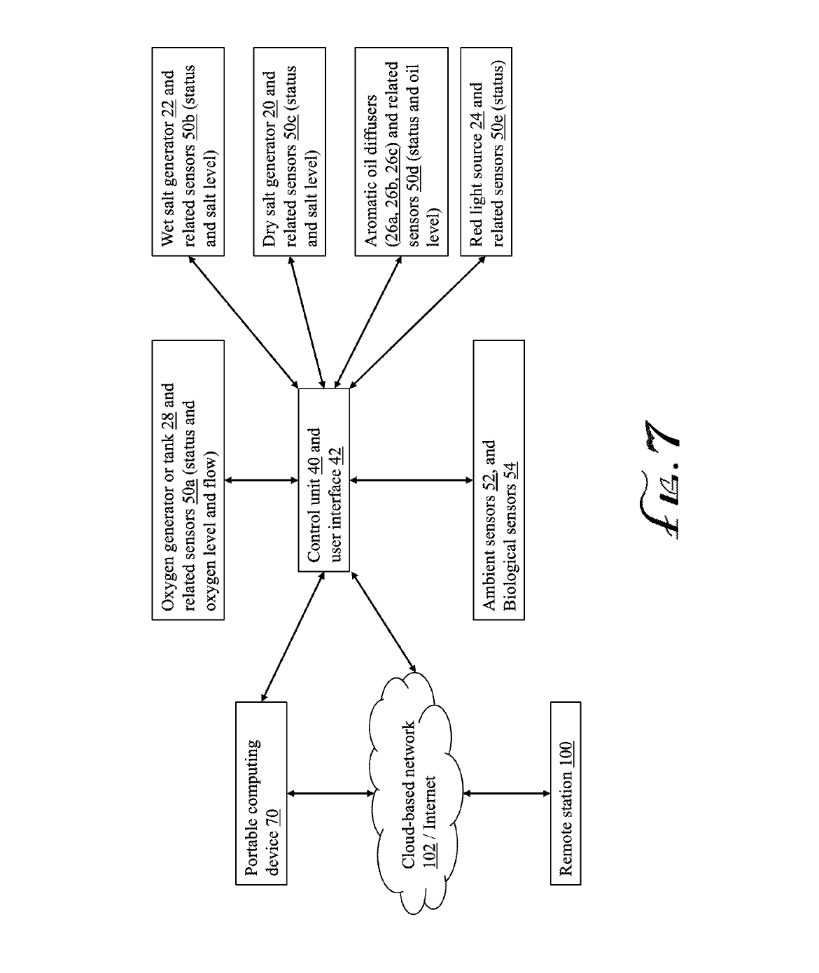

Referring to FIG. 7, the figure illustrates a block diagram showing interactions among the general modules and components of the capsule 10. As shown, all modules communicate with the control unit 40. The control unit 40 activates and controls functions of one or more modules, described above, within the capsule 10, and receives feedback and sensor inputs, as indicated by bidirectional arrows.

The control unit 40 communicates with: (1) the oxygen generator or tank 28 and related sensors 50a, including oxygen level, flow, and status sensors; (2) the wet salt generator 22 and related sensors 50b, including salt level and status sensors; (3) the dry salt generator 20 and related sensors 50c, including salt level and status sensors; (4) the aromatherapy module including aromatic oil diffusers 26a–26c, and related sensors 50d, including oil level and status sensors; (5) the red-light source 24 and related sensors 50e; (6) ambient sensors 52, including temperature, humidity, light, and air quality sensors, and biological sensors 54, including blood oxygen, blood pressure, pulse, and respiratory sensors; and (7) one or more portable computing devices 70, such as a desktop computer, smartphone, or tablet.

In exemplary embodiments, the sensors 50a–50e associated with the respective therapeutic modules are configured to provide operational feedback and status information to the control unit 40. Such sensors may include one or more status sensors configured to indicate whether a corresponding therapeutic module is active, inactive, enabled, disabled, or operating within a predetermined operational range. By way of example, a status sensor may indicate whether a dry salt generator, wet salt generator, oxygen delivery subsystem, aromatherapy diffuser, or red-light therapy module is currently energized, delivering therapy, in standby mode, or experiencing a fault condition. In addition to status sensing, the sensors 50a–50e may include functional sensors configured to monitor therapy-specific parameters, such as oxygen concentration or flow rate, saline or salt levels, aerosol output characteristics, light intensity or wavelength output, oil levels, or operational temperature of a module. The sensors 50a–50e may be physically coupled to, integrated within, or mounted adjacent to their corresponding therapeutic modules, including within housings, conduits, reservoirs, or delivery interfaces, to enable accurate monitoring of module performance. Sensor data generated by sensors 50a–50e is communicated to the control unit 40, which may use the data to regulate operation, adjust therapy parameters, generate alerts, or disable a module in response to detected conditions.

In exemplary embodiments, the ambient sensors 52 and biological sensors 54 may be positioned at one or more locations within or relative to the interior cabin 18 to facilitate monitoring of environmental conditions and user physiological parameters during a treatment session. Ambient sensors 52, including temperature, humidity, light, and air quality sensors, may be disposed within the interior cabin 18, mounted to interior surfaces such as walls, ceilings, panels, or airflow pathways, or positioned near air inlets or outlets to detect changes in environmental conditions within the enclosed space. Biological sensors 54 may be located within the interior cabin 18 at positions configured to obtain physiological measurements from a user, and may be integrated into the reclining seat 30, armrests, headrests, footrests, or other user-contact surfaces. In some implementations, one or more biological sensors 54 may be wearable, detachable, or selectively coupled to a user’s body, while remaining in communication with the control unit 40. The placement of ambient sensors 52 and biological sensors 54 enables real-time monitoring of the treatment environment and user response, allowing the control unit 40 to adjust therapeutic parameters, environmental conditions, or treatment sequencing to enhance safety, comfort, and therapeutic effectiveness.

The control unit 40 may include an embedded user-interface display, such as 42 as shown in FIG. 2. In some implementations, the user interface is provided through a portable computing device 70, such as a tablet or smart phone, which communicates with the control unit 40 to control and monitor functions of the capsule 10 remotely or locally.

The user interface may be provided through a software application, including Android-based, iOS-based, or web-based applications, configured to control and monitor functions of the capsule 10, including activation and deactivation of therapy modules, control of red-light wavelengths and intensity, salt concentration, aromatherapy diffusion, oxygen flow, heating, massage, and reclining functions. The user interface may further display sensor data, feedback, session reports, and historical treatment information.

In some implementations, the portable computing device 70 may be used by a user inside the capsule 10, or may be mounted or coupled to the interior cabin 18 to allow user interaction while seated on the reclining seat.

All interactions with the control unit 40, including user inputs, sensor data, treatment parameters, and session records, may be stored in a storage device operatively coupled to the control unit 40.

The control unit 40 is electrically connected to a power source, which may operate at one or more acceptable power ranges including, but not limited to, 120 volts alternating current (AC) at 60 hertz (120V/60Hz), 230 volts alternating current at 50 or 60 hertz (230V/50Hz or 230V/60Hz), or other regionally appropriate electrical standards. The power source may be provided through a conventional mains electrical supply, a dedicated electrical circuit, or an external power connection suitable for residential, commercial, or medical environments.

In exemplary embodiments, one or more additional modules of the capsule 10, including the therapeutic subsystems, sensors, actuators, lighting elements, heating elements, ventilation components, audio components, and user-interface devices, are also electrically supplied by the power source. Power distribution within the capsule 10 may include AC power, direct current (DC) power, or combinations thereof, and may further include internal power conversion components such as transformers, rectifiers, inverters, power supplies, voltage regulators, or isolation circuits configured to provide appropriate voltage, current, and electrical protection to each module.

In some implementations, one or more modules may be powered by low-voltage DC power, including but not limited to 12V, 24V, or 48V DC, while other modules may operate on AC power. The control unit 40 may manage or coordinate power distribution, activation, and shutdown of individual modules to enhance safety, energy efficiency, and system reliability.

In further implementations, the capsule 10 may include one or more energy storage devices, such as rechargeable batteries, battery packs, supercapacitors, or uninterruptible power supply (UPS) systems, configured to provide primary power, supplemental power, or backup power to the control unit 40 and/or selected modules. Battery-based power may enable portable operation, temporary off-grid use, controlled system shutdown, or continued operation during power interruptions. In such embodiments, the control unit 40 may be configured to monitor battery status, charging state, and power consumption, and to switch automatically between external power and stored energy as needed.

In further embodiments, and as additionally illustrated in FIG. 7, the control unit 40 is configured to communicate, either directly or indirectly through the portable computing device 70, with a remote station 100 via a network 102. The network 102 may comprise a cloud-based network, the Internet, a local area network (LAN), a wide area network (WAN), a cellular network, a wireless network, or combinations thereof. The remote station 100 may include one or more remote servers, cloud-based computing platforms, or centralized monitoring systems configured to receive operational data, sensor inputs, user selections, and treatment session records from the control unit 40.

In exemplary implementations, the remote station 100 is further configured to transmit control instructions, software updates, configuration settings, or treatment program data to the control unit 40, thereby enabling remote configuration, monitoring, diagnostics, and control of the capsule 10 from a location remote from the capsule. Such remote interaction may be performed by an authorized user, operator, technician, healthcare provider, or service administrator using a remote computing device.

The communication between the control unit 40, the portable computing device 70, and the remote station 100 may be bidirectional and may support real-time or near real-time data exchange. In some implementations, the remote station 100 enables centralized management of a plurality of capsules 10 deployed across multiple locations, facilitates data storage and analysis, supports predictive maintenance, and allows generation of reports related to system usage, treatment outcomes, and operational performance.

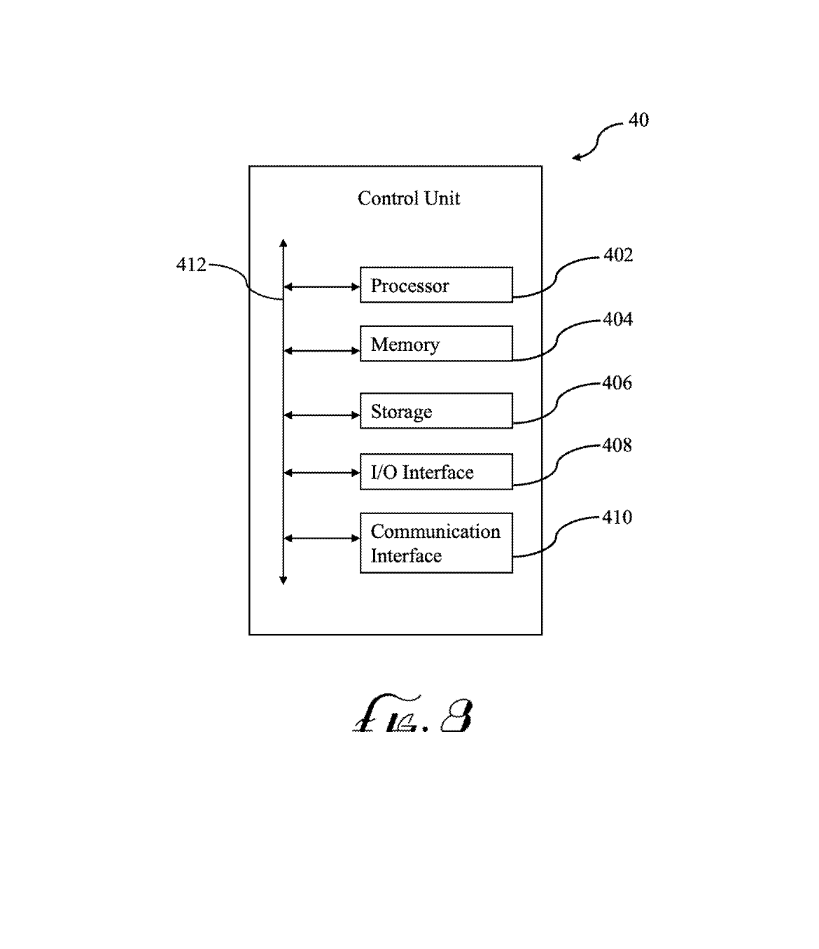

FIG. 8 illustrates an example computer system or control unit 40. In various embodiments of this invention, one or more control units 40 perform one or more steps of one or more methods or functions described or illustrated herein. In particular embodiments, software running on one or more computer systems or control units 40 performs one or more steps of one or more methods or functions described or illustrated herein or provides functionality described or illustrated herein. For example, software running on one or more control units 40 can perform one or more steps of one or more methods or functions described or illustrated herein in connection with the capsule 10. Herein, reference to the computer system or control unit may encompass a computing device, and vice versa, where appropriate. Moreover, reference to a computer system may encompass one or more computer systems/control units, where appropriate.

This disclosure contemplates control unit 40 taking any suitable physical form. As example and not by way of limitation, computer system or control unit 40 may be an embedded computer system, a system-on-chip (SOC), a single-board computer system (SBC) (such as, for example, a computer-on-module (COM) or system-on-module (SOM)), a desktop computer system, a laptop or notebook computer system, a mainframe, a mesh of computer systems, a server, a tablet computer system, or a combination of two or more of these. Where appropriate, computer system or control unit 40 may include one or more computer systems or control units 40; be unitary or distributed.

In particular embodiments, computer system or control unit 40 includes a processor 402, memory 404, storage 406, an input/output (I/O) interface 408, a communication interface 410, and a bus 412. Although this disclosure describes and illustrates a particular computer system or control unit having a particular number of particular components in a particular arrangement, this disclosure contemplates any suitable computer system having any suitable number of any suitable components in any suitable arrangement.

In particular embodiments, processor 402 includes hardware for executing instructions, such as those making up a computer program. As an example and not by way of limitation, to execute instructions, processor 402 may retrieve (or fetch) the instructions from an internal register, an internal cache, memory 404, or storage 406; decode and execute them; and then write one or more results to an internal register, an internal cache, memory 404, or storage 406. In particular embodiments, processor 402 may include one or more internal caches for data, instructions, or addresses. This disclosure contemplates processor 402 including any suitable number of any suitable internal caches, where appropriate. As an example and not by way of limitation, processor 402 may include one or more instruction caches, one or more data caches, and one or more translation lookaside buffers (TLBs). Instructions in the instruction caches may be copies of instructions in memory 404 or storage 406, and the instruction caches may speed up retrieval of those instructions by processor 402. Data in the data caches may be copies of data in memory 404 or storage 406 for instructions executing at processor 402 to operate on; the results of previous instructions executed at processor 402 for access by subsequent instructions executing at processor 402 or for writing to memory 404 or storage 406; or other suitable data. The data caches may speed up read or write operations by processor 402. The TLBs may speed up virtual-address translation for processor 402. In particular embodiments, processor 402 may include one or more internal registers for data, instructions, or addresses. This disclosure contemplates processor 402 including any suitable number of any suitable internal registers, where appropriate. Where appropriate, processor 402 may include one or more arithmetic logic units (ALUs); be a multi-core processor; or include one or more processors 402. Although this disclosure describes and illustrates a particular processor, this disclosure contemplates any suitable processor.

In particular embodiments, memory 404 includes main memory for storing instructions for processor 402 to execute or data for processor 402 to operate on. As an example and not by way of limitation, computer system or control unit 40 may load instructions from storage 406 or another source (such as, for example, another computer system or control unit 40) to memory 404. Processor 402 may then load the instructions from memory 404 to an internal register or internal cache. To execute the instructions, processor 402 may retrieve the instructions from the internal register or internal cache and decode them. During or after execution of the instructions, processor 402 may write one or more results (which may be intermediate or final results) to the internal register or internal cache. Processor 402 may then write one or more of those results to memory 404. In particular embodiments, processor 402 executes only instructions in one or more internal registers or internal caches or in memory 404 (as opposed to storage 406 or elsewhere) and operates only on data in one or more internal registers or internal caches or in memory 404 (as opposed to storage 406 or elsewhere). One or more memory buses (which may each include an address bus and a data bus) may couple processor 402 to memory 404. Bus 412 may include one or more memory buses, as described below. In particular embodiments, one or more memory management units (MMUs) reside between processor 402 and memory 404 and facilitate accesses to memory 404 requested by processor 402. In particular embodiments, memory 404 includes random access memory (RAM). This RAM may be volatile memory, where appropriate Where appropriate, this RAM may be dynamic RAM (DRAM) or static RAM (SRAM). Moreover, where appropriate, this RAM may be single-ported or multi-ported RAM. This disclosure contemplates any suitable RAM. Memory 404 may include one or more memories 404, where appropriate. Although this disclosure describes and illustrates particular memory, this disclosure contemplates any suitable memory.

In particular embodiments, storage 406 includes mass storage for data or instructions. As an example and not by way of limitation, storage 406 may include a hard disk drive (HDD), a floppy disk drive, flash memory, an optical disc, a magneto-optical disc, magnetic tape, or a Universal Serial Bus (USB) drive or a combination of two or more of these. Storage 406 may include removable or non-removable (or fixed) media, where appropriate. Storage 406 may be internal or external to computer system or control unit 40, where appropriate. In particular embodiments, storage 406 is non-volatile, solid-state memory. In particular embodiments, storage 406 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 406 taking any suitable physical form. Storage 406 may include one or more storage control units facilitating communication between processor 402 and storage 406, where appropriate. Where appropriate, storage 406 may include one or more storages 406. Although this disclosure describes and illustrates particular storage, this disclosure contemplates any suitable storage.

In particular embodiments, I/O interface 408 includes hardware, software, or both, providing one or more interfaces for communication between computer system or control unit 40 and one or more I/O devices. Computer system or control unit 40 may include one or more of these I/O devices, where appropriate. One or more of these I/O devices may enable communication between a person and computer system or control unit 40. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors or actuators. This disclosure contemplates any suitable I/O devices and any suitable I/O interfaces 408 for them. Where appropriate, I/O interface 408 may include one or more device or software drivers enabling processor 402 to drive one or more of these I/O devices. I/O interface 408 may include one or more I/O interfaces 408, where appropriate. Although this disclosure describes and illustrates a particular I/O interface, this disclosure contemplates any suitable I/O interface.

In particular embodiments, communication interface 410 includes hardware, software, or both providing one or more interfaces for communication (such as, for example, packet-based communication) between computer system or control unit 40 and one or more other computer systems or one or more networks. As an example and not by way of limitation, communication interface 410 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 410 for it. As an example and not by way of limitation, computer system or control unit 40 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system or control unit 40 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system or control unit 40 may include any suitable communication interface 410 for any of these networks, where appropriate. Communication interface 410 may include one or more communication interfaces 410, where appropriate. Although this disclosure describes and illustrates a particular communication interface, this disclosure contemplates any suitable communication interface.

In particular embodiments, bus 412 includes hardware, software, or both coupling components of computer system or control unit 40 to each other. As an example and not by way of limitation, bus 412 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 412 may include one or more buses 412, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

The foregoing descriptions of embodiments of the present invention have been presented only for purposes of illustration and description. They are not intended to be exhaustive or to limit the present invention to the forms disclosed. Accordingly, many modifications and variations will be apparent to practitioners skilled in the art. Additionally, the above disclosure is not intended to limit the present invention.

1. A treatment capsule for providing therapeutic treatment to a human user, comprising:
an enclosed cabin configured to position a human user inside the capsule;
a plurality of therapeutic modules coupled to the enclosed cabin, the plurality of therapeutic modules comprising a dry salt generator and a wet salt generator; and
a control unit operatively coupled to the plurality of therapeutic modules,
wherein the control unit is configured to selectively control operation of one or more of the therapeutic modules during a treatment session.

2. The treatment capsule of claim 1, wherein the plurality of therapeutic modules further comprising a red-light therapy module.

3. The treatment capsule of claim 2, wherein the plurality of therapeutic modules further comprising an aromatherapy module and an oxygen delivery module.

4. The treatment capsule of claim 1, wherein the therapeutic modules are operable independently and in coordinated sequences under control of the control unit.

5. The treatment capsule of claim 1, further comprising one or more sensors operatively coupled to the control unit and configured to provide feedback indicative of at least one operating condition and at least one environmental parameter within the enclosed cabin.

6. The treatment capsule of claim 5, wherein the sensors comprising temperature sensors, humidity sensors, air quality sensors.

7. The treatment capsule of claim 1, further comprising a user interface operatively coupled to the control unit and configured to receive user input relating to operation of the therapeutic modules.

8. The treatment capsule of claim 7, wherein the user interface is provided by a portable computing device in communication with the control unit.

9. A treatment capsule for providing salt-based therapeutic treatment to a human user, comprising:
an enclosed cabin configured to position a human user inside the capsule;
a dry salt generator configured to deliver dry salt aerosol into the enclosed cabin;
a wet salt generator configured to deliver a saline aerosol into the enclosed cabin; and
a control unit operatively coupled to the dry salt generator and the wet salt generator,
wherein the control unit is configured to operate the dry salt generator and the wet salt generator selectively, sequentially, and simultaneously during a treatment session.

10. The treatment capsule of claim 9, wherein the dry salt generator and the wet salt generator are fluidly coupled to the enclosed cabin through respective conduits terminating in outlet nozzles disposed within the enclosed cabin.

11. The treatment capsule of claim 9, further comprising an air ventilator configured to control airflow within the enclosed cabin during delivery of salt aerosols.

12. The treatment capsule of claim 9, wherein the control unit is configured to regulate salt concentration, aerosol flow rate, and treatment duration.

13. The treatment capsule of claim 9, further comprising an aromatherapy module configured to introduce aromatic substances into the enclosed cabin.

14. The treatment capsule of claim 13, wherein the aromatherapy module includes a plurality of aromatic oil diffusers independently controllable by the control unit.

15. The treatment capsule of claim 9, further comprising a red-light therapy module configured to emit red light and near-infrared light into the enclosed cabin.

16. The treatment capsule of claim 15, wherein the control unit is configured to control wavelength, intensity, and duration of light emitted by the red-light therapy module.

17. A treatment capsule for providing coordinated therapeutic treatment to a human user, comprising:
an enclosed cabin configured to position a human user inside the capsule;a plurality of therapeutic subsystems comprising:a dry salt therapy subsystem;a wet salt therapy subsystem;a red light therapy subsystem;an aromatherapy subsystem;an oxygen therapy subsystem; anda control unit operatively coupled to each of the therapeutic subsystems,wherein the control unit is configured to coordinate operation of two or more of the therapeutic subsystems during a single treatment session.

18. The treatment capsule of claim 17, wherein the control unit is configured to deliver at least two therapeutic subsystems simultaneously within the enclosed cabin.

19. The treatment capsule of claim 17, wherein the control unit is configured to execute a predefined and user-customizable treatment program comprising sequential activation of the therapeutic subsystems.

20. The treatment capsule of claim 17, wherein the control unit is configured to communicate with a remote station via a network to enable remote monitoring, configuration, and control of the treatment capsule.
